(19) United States
(12) Patent Application Publication
(10) Pub. No.: US 2015/0157349 A1
(43) Pub. Date: Jun. 11, 2015

(54) SURGICAL FORCEPS

(71) Applicant: OLYMPUS WINTER & IBE GMBH, Hamburg (DE)

(72) Inventor: Simon Hirschfeld, Hamburg (DE)

(21) Appl. No.: 14/415,244

(22) PCT Filed: Jul. 17, 2013
(86) PCT No.: PCT/EP2013/002128

§ 371 Date: Jan. 16, 2015

(30) Foreign Application Priority Data
Aug. 21, 2012 (DE) 10 2012 016 396.7

(51) Int. Cl. A61B 17/29

(52) U.S. Cl. CPC A61B 17/2909; A61B 2017/292; A61B 2017/293; A61B 2017/2931; A61B 2017/2946; A61B 2019/4868

(57) ABSTRACT

Surgical forceps with a shaft at the distal end of which a jaws piece including jaws is removably mounted. The jaws piece is connected in a rotationally fixed manner to the distal end of an actuating rod running parallel to the shaft. The proximal end of the actuating rod can be coupled in a rotationally fixed and longitudinally displaceable manner to a handle arranged on the proximal end of the shaft for transferring movements executed in the longitudinal direction of the shaft. The surgical forceps are characterized in that the handle is designed in such a manner that the rotationally fixed coupling to the actuating rod disengages when the handle is actuated to open the jaws piece.

The invention relates to surgical forceps of the kind specified in the preamble of claim 1.

Generic forceps are known from DE 94 18 094 U1. They can be provided with a longer shaft so that they can be used in endoscopic surgery or laparoscopy. Such forceps can in particular also be used as needle holders which can be used for suturing in endoscopic surgery.

Such forceps can be disassembled for cleaning purposes. To this end, the jaws piece is removed from the distal end of the shaft. Conventional connections at this point are screw threads or bayonet couplings. Such connections can be unintentionally released during torsional load, which occurs frequently during suturing, and therefore require additional rotation prevention, for which no space is available in the distal end region of the shaft. Generically, the proximal end of the actuating rod, which is connected to the jaws piece in a rotationally fixed manner, is therefore coupled in a rotationally fixed manner. For this, sufficient space is available in the region of the handle. The rotationally fixed coupling has to permit longitudinal displacements of the actuating rod, thus the working movement thereof.

In the case of the known generic construction, the rotationally fixed coupling of the actuating rod is carried out at a square portion of the actuating rod by means of clamping jaws which engage there and which are controlled by a sliding sleeve that is mounted to be longitudinally displaceable on the handle.

Thus, the known construction needs additional manually actuatable elements for the rotationally fixed coupling of the actuating rod. It is a constructionally very complex design and requires additional operating steps for the actuation thereof.

It is an object of the present invention to simplify generic forceps with regard to the construction and handling thereof.

This object is achieved with the features of the characterizing part of claim 1.

According to the invention, the handle is designed in such a manner that the rotationally fixed coupling disengages when the handle moves in the direction of the open position of the jaws. Thus, the handle has simply to be actuated for opening the jaws so as to thereby also automatically disengage the rotationally fixed coupling of the actuating rod. Additional actuations of the rotary coupling are not required. This also allows simplifying the construction of the forceps significantly. This construction ensures that the rotationally fixed coupling engages upon closing the forceps, and unintentional disengaging of the jaws pieces during any surgical work with the forceps is prevented. The jaws piece can only be removed when it is opened. This construction results in high degree of safety for the patient while providing easy operability.

An embodiment according to claim 2, which is advantageous with regard to its constructional simplicity, provides that a movable handle piece of the handle engages with the actuating rod in a rotationally locked manner.

According to claim 3, a rotation-preventing form-fitting engagement is advantageously ensured via a movement region of the handle piece, which form-fitting engagement, e.g., is advantageously provided according to claim 4 on the movable handle piece by a fork that can engage in a rotation-preventing manner via parallel surfaces on the actuating rod. Thus, the longitudinal movability of the actuating rod is maintained in the required manner during the rotation-preventing engagement.

The invention is exemplary and schematically illustrated in the drawing. In the figures:

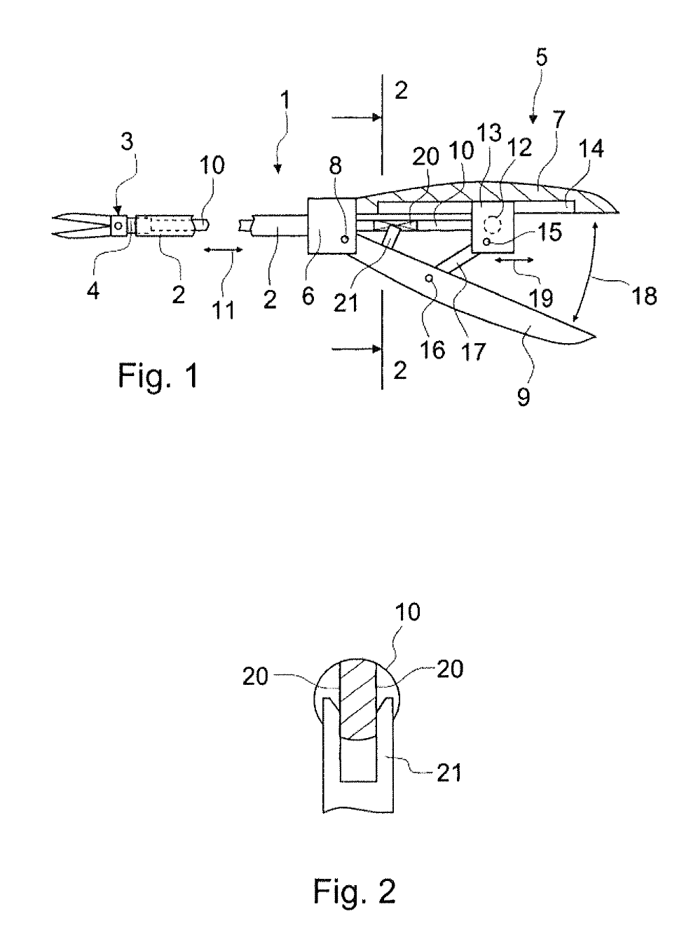

FIG. 1 shows a side view of surgical forceps according to the invention with an actuating rod,

FIG. 2 shows a section along line 2 in FIG. 1 in the region of the actuating rod,

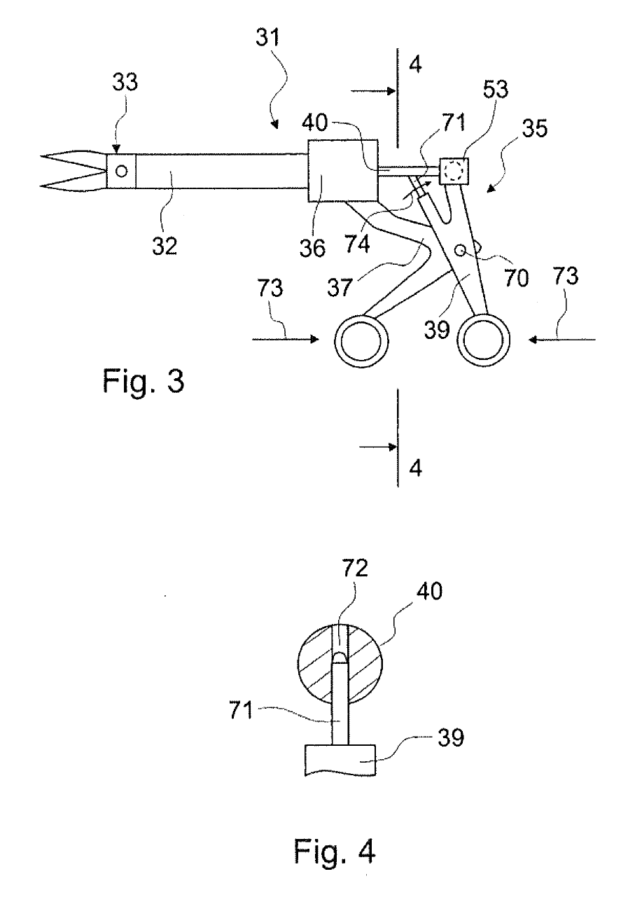

FIG. 3 shows a side view of surgical forceps of another embodiment, with an actuating rod, and

FIG. 4 shows a section along line 4 - 4 in FIG. 3 in the region of the actuating rod.

FIGS. 1 and 2 show a first embodiment of surgical forceps 1, which is provided with an elongated tubular shaft 2, at the distal end of which a jaws piece 3 forming the illustrated forceps jaws can be screwed in with a thread 4, which is shown partially unscrewed in FIG. 1.

A handle piece 5 is fastened, e.g. in a removable manner, with its main body 6 to the proximal end of the shaft 2.

The handle 5 illustrated in FIG. 1 is has an elongated design, thus extends substantially in the direction of the shaft 2.

The handle 5 has two handle pieces, namely a fixed handle piece 7 stationarily arranged on the main body 6, and a movable handle piece 9 pivotably mounted on the main body 6 by means of a pivot pin 8. The handle pieces 7 and 9 can be grasped by a hand and can be pressed together from the illustrated partially opened position.

An actuating rod 10, which causes the jaws of the jaws piece 3 to open and close upon longitudinal actuation in the direction of the arrow 11, is connected to the jaws piece 3 in a rotationally fixed manner. The proximal end region of the actuating rod 10 protrudes beyond the main body 6 up into the region between the handle pieces 7 and 9 and is detachably fastened there in a coupling body 13 by means of an end body 12 illustrated as a dashed ball, which coupling body is guided in a longitudinal guiding member, illustrated as groove 14 in FIG. 1, on the fixed handle piece 7 to be longitudinally displaceable in the direction of the shaft 2.

A coupling rod 17, which is mounted with pivot pins 15 and 16 on the coupling body 13 and the movable handle piece 9, ensures that during the opening and closing movement of the handle pieces 7 and 9 in the direction of the arrow 18, the coupling body 13 is moved in the direction of the arrow 19, thus in the direction of the shaft 2. In the process of this, the coupling body drives the coupled actuating rod 10 in the direction of the arrow 11.

When closing the handle 5, thus when the handle pieces 7 and 9 approach one another, the actuating rod 10 is retracted through the illustrated kinematics, and the jaws of the jaws piece 3 are therefore closed.

The illustrated construction is designed to be robust enough that it can be used as a needle holder and that it is able to exert the forces required for this purpose. The length of the shaft 2 can be selected to be long enough for the forceps to be usable for endoscopic or laparoscopic purposes. Thus, the forceps can in particular be used for endoscopic suturing in the abdominal cavity.

In particular when used as a needle holder, the illustrated forceps has also to be provided with a safety catch that locks the forceps in the closed state. Said safety catch can usually be formed between the handle pieces 7 and 9. Such a locking construction is omitted in the illustrated exemplary embodiment for reasons of simplifying the drawing.

Furthermore, an expander spring which is not illustrated and which forces the non-actuated handle 5 into the open position can be arranged between the handle pieces 7 and 9.

The illustrated connection between the jaws piece 3 and the shaft 2 by means of a thread 4 is not prevented from rotating. If the jaws piece 3 engages fixedly at any body site and the physician tries to twist the forceps by applying force, for example, in order to pierce a needle with a rotary movement into very hard tissue, untightening of the thread 4 can occur. The jaws piece 3 loosens and in the extreme case, it can fall off and remain in the body. Less dramatic cases are still very disturbing if, e.g., a gap forms in the region of the thread 4 between the jaws piece 3 and the shaft 2, where contaminants can accumulate. Bayonet coupling also pose the risk of loosening due to rotation.

Thus, generic forceps such as the one illustrated in the aforementioned document need additional rotation prevention, for which no space is available in the distal end region of the shaft 2. Therefore, rotation prevention for the actuating rod 10 is provided in the region of the handle 5.

For this proximal rotation prevention of the actuating rod 10, the actuating rod has a shaped portion at which the actuating rod has the cross-section which is shown hatched in FIG. 2 and which is delimited by two parallel surfaces 20. A shaped piece in the form of a fork 21 provided with the two fork arms illustrated in FIG. 2 is fastened on the movable handle piece 9. The fork has a width, thus a spacing between the fork arms, that corresponds to the spacing between the parallel surfaces 20. As shown in FIG. 2, the fork 21 thus can engage with the actuating rod 10 in the shaped portion thereof having the surfaces 20 in a rotationally fixed and form-fitting manner. The longitudinal displaceability of the actuating rod 10 is still maintained.

The fork 21 is arranged on the movable handle piece 9 in such a manner that when the handle closes, thus when the movable handle piece 9 moves towards the fixed handle piece 7, the fork engages with the actuating rod. With the handle completely open, thus when the movable handle piece 9 is moved far away from the fixed handle piece 7, the fork 21 disengages from the actuating rod 10. The actuating rod 10 can then be rotated, e.g. for unscrewing the jaws piece 3 from the distal end of the tubular shaft 2 in order to disassemble the forceps.

FIGS. 3 and 4 show a surgical forceps 31 that differs from the embodiment of FIG. 1 substantially by the angled shape of the handle 35.

The forceps 31 illustrated in FIG. 3 corresponds to the construction of FIG. 1 with respect to the shaft 32 and the jaws piece 33. Thus, the jaws piece 33 is also connected here to the tubular shaft 32 by a connection susceptible to rotary movements, thus by a thread or a bayonet coupling, for example, and requires additional rotation prevention at the proximal end of the actuating rod 40.

The actuating rod 40 is coupled at its proximal end to the coupling body 53 which, in the embodiment of the handle 35 illustrated in FIG. 3, is located at the end of a movable handle piece 39. A main body 36, on which a fixed handle piece 37 is fastened, is located at the proximal end of the shaft 32. The movable handle piece 39 is pivotably mounted on said fixed handle piece by means of an articulated joint 70. Conventionally designed finger rings, which can be gripped with two fingers of a hand, are fastened to the free ends of the handle pieces 37 and 39.

A pin 71 as a shaped piece is fastened on the movable handle piece 39. A shaped portion on the actuating rod 40 is provided with a longitudinal slot 72.

By pressing the free ends of the handle pieces 37 and 39 together as indicated by the arrows 73, the actuating rod 40 retracts and the jaws of the jaws piece 33 are closed. In the process of this, the movable handle piece 39 is pivoted clockwise about the articulated joint 70. Thereby, the pin 71 moves in the direction of the arrow 74. During this movement, the pin reaches the region of the actuating rod 40 and extends into the longitudinal slot 72 thereof so that a rotation-preventing form-fitting engagement is obtained between the pin 71 and the longitudinal slot 72.

For opening the forceps, the movable handle piece 39 has to be pivoted counterclockwise. In the process of this, the pin 71 moves out of the longitudinal slot 72 and the rotation prevention is released.

The embodiment of the FIGS. 3 and 4 can also be designed as a needle holder. The special type of rotation prevention of FIG. 2 can also be used for the special shape of the handle according to FIG. 3 and, vice versa, the special rotation prevention of FIG. 4 can also be used for the handle of FIG. 1.

1. A surgical forceps having a shaft at the distal end of which a jaws piece comprising jaws is removably fastened, said jaws piece being connected in a rotationally fixed manner to the distal end of an actuating rod running parallel to the shaft, wherein the proximal end of said actuating rod is connected to a handle arranged on the proximal end of the shaft and can be coupled thereto in a rotationally fixed and longitudinally displaceable manner for transferring movements performed in the longitudinal direction of the shaft, wherein the handle is designed in such a manner that its rotationally fixed coupling disengages from the actuating rod when the handle is actuated for opening the jaws piece.

2. The forceps according to claim 1, wherein the handle has a handle piece which is movable for actuating the jaws piece and which, in its position closing the jaws piece, engages with the actuating rod in rotationally locking manner.

3. The forceps according to claim 2, wherein the movable handle piece is arranged and designed in such a manner that when actuated in the direction of closing the jaws piece, it approaches the actuating rod with a shaped piece which within the movement region of the movable handle piece is in rotation-preventing engagement with a shaped portion of the actuating rod, said shaped portion being designed for engaging.

4. The forceps according to claim 3, wherein the shaped piece is designed as a fork and that the shaped portion has surfaces that are parallel to the axis of the actuating rod and are spaced apart corresponding to the fork width.
